(19) United States
(12) Patent Application Publication
(10) Pub. No.: US 2011/0142911 A1
(43) Pub. Date: Jun. 16, 2011

(54) HIV epitopes and pharmaceutical composition containing same

(75) Inventors: Yu-Chun Lone, Paris (FR); Anthony Pajot, Paris (FR)

(73) Assignee: Centre National de la Recherche Scientifique (CNRS), Paris (FR)

(21) Appl. No.: 11/894,172

(22) Filed: Aug. 20, 2007

Related U.S. Application Data
(63) Continuation of application No. PCT/FR2006/050167, filed on Feb. 24, 2006.

(30) Foreign Application Priority Data
Feb. 25, 2005 (FR) 0550520

(51) Int. Cl. A61K 9/127; C07K 14/005; C07K 16/10; C07H 21/00; A61K 31/7088; A61K 35/12; A61K 39/21; C12N 5/078; G01N 33/53; C12N 5/0784; C12N 5/0786; A61P 37/04; A61P 31/18

(52) U.S. Cl. 424/450; 530/350; 530/387.9; 530/326; 536/23.1; 514/44 R; 424/93.71; 424/185.1; 435/325; 435/7.24

(57) ABSTRACT

The invention concerns a peptide comprising an epitope consisting of a sequence of amino acids selected among SEQ ID NO 1, SEQ ID NO 2, SEQ ID NO 3, and having a length not more than 24 amino acids.

The present invention concerns epitopes derived from viral antigens capable of specifically binding to HLA-DR1 molecules and of inducing an immune response in subjects with HLA-DR1 phenotypes, as well as their functional analogs.

The present invention also relates to pharmaceutical compositions containing peptides comprising these epitopes and/or their functional analogs, as well as to diagnostic methods for determining the immune condition of an individual likely to have a viral infection, comprising the use of these peptides.

More specifically, the present invention relates to immunogenic epitopes derived from an antigen of the human immunodeficiency virus (HIV), the protein p24, consisting in a sequence of amino acids selected from SEQ ID NO 1, SEQ ID NO 2, SEQ ID NO 3.

HIV type viruses are viruses causing chronic infection, i.e., not inducing a sufficient immune response for eliminating the infection.

This infection may result in the appearance of an acquired immunodeficiency syndrome (AIDS). This disease is notably characterized by increased sensitivity to infections by opportunistic pathogens, or by the appearance of aggressive forms of Kaposi's sarcoma, or the appearance of B cell lymphomas, associated with a significant reduction in the number of CD4+ T cells.

Many vaccine strategies have been developed in order to combat this infection. Most of these strategies resort to means aiming at improving the production of antibodies in order to prevent infection in the case of exposure to the HIV, or to means aiming at activating cytotoxic T lymphocytes (or CD8+ or CTL), in order to detect and destroy the infected cells, and thereby control and eliminate the infection.

Various proteins of the viral capsid of the HIV have been used in order to produce antibodies. Thus, the use of peptides of the p24 antigen of the Gag protein of the HIV for producing antibodies is known from U.S. Pat. No. 6,593,079.

It appears that the response depending on the CD4+ T cells or helper T lymphocytes (or HTL) significantly intervenes in maintaining adequate CD8+ T cell functions and in controlling viremia.

However, CD4+ T cells specifically responding to the HIV appear to be preferentially infected and eliminated by the HIV, thereby limiting the capacity of potential vaccines of activating helper T cells after the onset of the infection (Douek et al., Nature, 2002, 417:95-98). Chronically infected patients are unable to trigger a T cell type response both against antigens of the HIV and against antigens of opportunistic pathogens. Thus, loss of the HTL response plays a central role when developing an HIV infection.

In spite of the central role of these cells in controlling HIV infection, only a small number of restricted immunogenic CD4+ epitopes have been identified up to now (Wilson et al., J. Virol, 2001, 75:4195-207; Kaufmann et al., J. Virol., 2004, 78:4463-77).

Thus, there is a need for new CD4+ restricted epitopes with which a response depending on the CD4+ T lymphocytes directed against antigens of the HIV may be activated.

Further there is a need for having pharmaceutical compositions, notably vaccines, intended to prevent and/or treat HIV infections.

Moreover, it is important to be able to describe the immune response and/or the immune condition of a patient likely to be affected by an HIV infection so as to be able to determine his/her sensitivity to the disease and to better adjust the treatment to be administered.

Thus, there is a need for having a marker with which the immune condition of a patient may be characterized.

There also is a need for having a diagnostic method with which the immune condition of a subject likely to be affected by an HIV infection may be described, as well, if need be, for evaluating the response of his/her immune system to a treatment.

The object of the present invention is to notably meet these needs. The inventors have identified three epitopes derived from the p24 antigen stemming from the Gag protein of the HIV, specific to the HLA-DR1 phenotype.

According to one of its first aspects, the present invention relates to a peptide comprising at least one epitope consisting in a sequence of amino acids selected from SEQ ID NO 1, SEQ ID NO 2, SEQ ID NO 3, and functional analogs thereof.

The inventors have observed that these epitopes were capable of inducing proliferation in vitro of CD4+ T cells stemming from class II (IAβ^{b0}) H-2 invalidated HLA-DR1 transgenic mice (Pajot et al., Int. Immunol., 2004, 16:1275-1282) vaccinated by means of the p24 protein. Moreover, these peptides are capable of inducing a CD4+ T cell type response of cells from patients affected by the HIV, notably by the HIV-1, or from seronegative individuals, following activation of these cells by antigen presenter cells. The proliferating CD4+ T cells in response to the peptides according to the invention are characterized by an HLA-DR1 phenotype and the biological response is characterized by secretion of a cytokine, IFN-γγ, as measured by ELISPOT.

In the sense of the present invention, the term “functional analogs” is intended to designate any peptide or peptide analog capable of mimicking immunogenic activity of the peptides according to the invention, i.e. notably having the capacity of binding to HLA-DR1 molecules and inducing a biological response in CD4+ T lymphocytes.

HLA means “Human Leukocyte Antigen” and is equivalent to MHC, “Major Histocompatibility Complex”. HLAs (or MHCs) correspond to a set of genes divided into Classes I and II, having very high polymorphism, and coding for molecules, the function of which is to present peptide fragments at the surface of the cells.

In the sense of the present invention, “immunogen” is intended to designate any substance capable of causing an immune response. An immunogen or immunogenic substance is functionally distinct from an antigen. An antigen is defined as being any substance capable of binding to a specific antibody. Therefore, all antigens are considered as being able to induce a response of the antibody type, but some of them must be bound to an immunogen in order to be able to act in this way. Thus, although all immunogens are antigens, all antigens are not immunogenic.

According to another of its aspects, the present invention relates to a peptide comprising an epitope consisting in a sequence of amino acids selected from SEQ ID NO 1, SEQ ID NO 2, SEQ ID NO 3, and functional analogs of the latter, and having a length smaller than or equal to 24 amino acids.

According to another of its aspects, the present invention relates to a multi-epitope peptide comprising at least one epitope consisting in a sequence of amino acids selected from SEQ ID NO 1, SEQ ID NO 2, SEQ ID NO 3, and functional analogs thereof.

According to still another object, the present invention relates to the use of at least one peptide according to the invention and/or one nucleic acid coding for said sequence in order to prepare a pharmaceutical composition intended to prevent and/or treat an infection by a human immunodeficiency virus in patients of HLA-DR1 phenotype.

Still according to another object, the invention relates to a peptide according to the invention associated with a biotinylated complex consisting of an α chain (DRα) and a β chain (DRβ) of an HLA of type II, of a spacer arm and streptavidin in order to form a Class II tetramer.

According to still another of its aspects, the present invention relates to a pharmaceutical composition containing at least one effective amount of at least one peptide according to the invention, in combination with a pharmaceutically acceptable carrier, excipient, diluent and/or adjuvant.

Peptides

The peptides according to the invention are isolated peptides comprising at least one epitope consisting in a sequence of amino acids selected from SEQ ID NO 1, SEQ ID NO 2, SEQ ID NO 3, and functional analogs thereof.

In the sense of the present invention, by “peptide” is meant a chain of amino acids connected by means of peptide bonds and which may comprise amino acids of the D or L type, and also any derivative of amino acids, such as citrulline, ornithine, β-aminobutyric acid, as well as non-natural amino acids, which would suit the invention. Usually, a peptide may comprise 2 to 30 amino acids, in particular 6 to 25, and notably 12 to 20 amino acids.

In the sense of the present invention, by “epitope” is meant a region of an antigen recognized by an antigen or a receiver for antigens. The epitopes are also designated as “antigenic determinant”. A T cell epitope is a short peptide (from 6 to 10 amino acids for CD8+ T cells, and from 12 to 24 amino acids for CD4+ T cells) derived from a proteinaceous antigen.

In particular, peptides according to the invention are isolated peptides comprising an epitope consisting in a sequence of amino acids selected from SEQ ID NO 1, SEQ ID NO 2, SEQ ID NO 3, and functional analogs thereof and having a length less than or equal to 24 amino acids.

More particularly, peptides according to the invention are isolated peptides consisting in a sequence of amino acids selected from SEQ ID NO 1, SEQ ID NO 2, SEQ ID NO 3, and functional analogs thereof.

Functional analogs or equivalent biological functional analogs, are understood by one skilled in the art as directed at peptides comprising structural changes as compared with the peptides according to the invention but on the other hand retaining immunogenic features (or functional biological properties) of the peptides according to the invention, which are defined by the capability of interaction of these peptides with the HLA-DR1 molecules and induction of a biological response in CD4+ T lymphocytes. The various conceivable changes may be introduced by physical, chemical and/or biological means, such as for example substituting one or more amino acids with other ones, inserting one or more amino acids and/or deleting one or more amino acids.

According to an advantageous alternative, the functional analogs of peptides according to the invention are selected from those having improved functional biological properties.

Because of their relatively small size, the peptides according to the invention may be obtained by any peptide synthesis technique, known to one skilled in the art, such as for example methods of synthesis in solution or synthesis methods in a solid phase.

Apart from peptide synthesis, obtaining the peptides according to the invention by other methods, notably by recombinant DNA techniques, may also be contemplated, as described later on.

According to another embodiment, the peptides of the present invention or analogs thereof may also be associated with other peptides in order to increase their immunogenic properties. For example, the peptides according to the invention may be associated, notably by means of a spacer arm, with other peptides comprising epitopes with e.g. a stimulating activity with regard to the response of cytotoxic T lymphocytes. As an example of a peptide comprising epitopes derived from HIV antigens capable of activating a cytotoxic T lymphocyte response, the peptides mentioned in application WO 00/29008 may be mentioned.

The spacer arm may comprise molecules of relatively small sizes, such as amino acids or the like, and which are substantially neutral under physiological conditions.

When it is present, the spacer arm may comprise at least one or two amino acids, either identical or different, and notably three to fifteen amino acids, and in particular six to ten amino acids.

According to another alternative, the peptides according to the invention may be connected to another peptide without any spacer arm.

According to another embodiment of the invention, at least one of the peptides according to the invention may be comprised in a multi-epitope peptide construct.

A polypeptide or peptide or poly-epitope or multi-epitope protein may for example comprise 2 to 50 immunogenic peptides combined into a single polypeptide by means of recombinant or synthesis techniques, notably 4 to 20, and more particularly 8 to 12 immunogenic peptides.

The multi-epitope peptides may be linear or branched. They may be produced by means of chemical synthesis methods of by means of a recombinant DNA technique.

The multi-epitope construction may be of the heteropolymer or homopolymer type.

It may comprise epitopes stimulating the activity of helper T lymphocytes and/or cytotoxic T lymphocytes.

The peptides according to the invention are advantageously used as such or formulated in a pharmaceutical composition as for example defined in the following for preventing and/or treating an HIV infection, and in particular an HIV-1 infection.

Nucleic Acids and Expression Vectors

One skilled in the art in order to obtain peptides according to the invention according to recombinant DNA based methods, may for example use the manual “Molecular Cloning—a Laboratory Manual” (2^{nd} edition), Sambrook et al., 1989, vol. 1-III, Coldspring Harbor Laboratory, Coldspring Harbor Press, NY, (Sambrook).

Because of degeneration of the genetic code, it is appreciated that with many variations preserving constructs of nucleic acids, it is possible to obtain sequences of functionally identical amino acids.

The consequence of introducing a change in a sequence of nucleic acids according to the invention may notably be evaluated by determining the immunogenic properties of the peptides coded by the sequences of nucleic acids by means of any known immunological tests, and possibly comprising the comparison of the results obtained with changed peptides, with those obtained with unchanged peptides.

Obtaining the multi-epitope constructs mentioned earlier may be performed by ligature of recombinant or synthetic nucleic acid sequences coding for each of the peptides comprising an epitope, either by enzymes (for example of the ligase type) or by chemical synthesis.

Thus, the present invention relates to sequences of nucleic acids coding for at least one peptide according to the invention, as well as for their functional analogs.

Still according to one embodiment, at least one of the sequences of nucleic acids according to the invention may be introduced into any suitable expression vector.

These vectors may subsequently be used for transforming the host cells and producing the desired recombinant peptides.

In order to allow expression of at least one recombinant peptide in the host cells, the sequence of nucleic acids coding for the latter may be functionally bound in an expression vector at its start and stop codon, respectively, with a promoter region and a terminator region and usually a replication system.

The use of host cells, such as bacterial, yeast, insect, mammalian or plant cells for producing peptides according to the invention by using the expression vectors defined earlier may also be contemplated within the scope of the present invention.

Advantageously, the mammalian cells in which it is possible to express an expression vector comprising a nucleic acid according to the invention, may for example be antigen presenter cells.

The expression vectors containing the sequences of nucleic acids coding for the peptides according to the present invention may be used for transforming host cells by any suitable technique, such as for example transfection with calcium phosphate or electroporation.

Antibodies

According to another embodiment, the present invention also relates to at least one antibody which may be obtained by means of at least one peptide according to the present invention. This antibody has the capability of specifically binding to an epitope consisting in a sequence of amino acids selected from SEQ ID NO 1, SEQ ID NO 2, SEQ ID NO 3.

Thus, the peptides according to the invention may be used for producing antibodies by means of different techniques known to one skilled in the art (such as those described in Current Protocols in Immunology, Willey/Greene NY; and Antibodies: Laboratory Manual Harlow, Harlow and Lane, Cold Spring Harbor Laboratory Press, 1989).

The thereby obtained antibodies may be used for example as diagnostic and/or therapeutic tools in individuals likely to be affected by an HIV infection.

The peptides according to the invention may be used as such for obtaining monoclonal or polyclonal antibodies, or they may be associated with other immunogens, for example with other fragments of bacterial proteins, having immunogenic properties such as hemagglutinin A of the influenza virus, or with other epitopes in multi-epitope peptide constructs or in association with an HLA-DR1 molecule or a fragment thereof.

According to an embodiment, an antibody according to the invention may be of a specific complex type and specifically bind to a complex comprising an epitope according to the invention and an HLA-DR1 molecule.

The antibodies according to the invention comprise antibody fragments with the capability of binding to the peptides according to the invention (for example the Fab fragments), as well as the antibodies of the recombinant single chain type (for example scFv antibodies).

The monoclonal antibodies may be obtained from cells secreting the desired antibody, such as B lymphocytes in culture or hybridomas.

Tetramers

According to still another embodiment, the peptides according to the invention may be used for preparing tetramers.

The preparation and use of tetramers is well known to one skilled in the art (see notably, Novak et al., J. Clin. Invest., 1999, 104:R63-R67; Cochran et al., Immunity, 2000, 12:241-250).

A tetramer comprising a peptide according to the invention may be obtained in the following way: corresponding α and β heavy chains of the HLA-DR1 molecule are folded in the presence of peptides capable of binding thereto in order to generate complexes.

According to another embodiment, it is possible to use a peptide bound by genetic engineering to the N-terminal end of the DRβ chain as detailed by Iyasere at al., (J. Viral., 2003).

The complex is then biotinylated at the C-terminal end of the DRβ heavy chain of the HLA-DR1 molecule, including a biotinylation site for example added by recombinant DNA techniques. Formation of the tetramer is then induced by adding streptavidin.

Both DRα and DRβ heavy chains of the HLA-DR1 molecule may be produced either in insect cells, for example in Drosophila cells, and connected for example by a Leu Zipper domain, and by a biotin-streptavidin-PE (phycoerythrin) interaction (Novak at al., J. Clin. Invest., 1999) i.e. produced in bacteria, for example in Escherichia coli (Cochran J R et al., Immunity, 2000, 12:241-250).

When streptavidin marked by means of a fluorescent probe is used, the tetrameric complex may be used for identifying specific cells of an antigen, for example by means of flux cytometry.

Such methods may be used for purposes of diagnosing and/or prognosing the immune condition of an individual of HLA-DR1 phenotype, before, during and/or after a therapeutic and/or prophylactic treatment.

The cells identified by means of the methods described earlier may be used for therapeutical purposes, notably after in vitro amplification and injection into an individual.

Cells

According to an embodiment, clones of helper (or CD4+) T lymphocytes isolated and activated by means of the peptides according to the present invention or functional analogs thereof, may be used as a prophylactic and/or therapeutic agent with regard to an HIV infection, and in particular to an HIV-1 infection.

Thus, according to one of its objects, the present invention also relates to at least one clone of an isolated helper T lymphocyte comprising a receptor specifically recognizing a complex containing an HLA-DR1 molecule and a peptide according to the invention.

The present invention further relates to a method for treating and/or preventing an infection by a human immunodeficiency virus, comprising at least one step consisting of administering to an individual an effective amount of at least one helper T lymphocyte clone according to the invention.

In the sense of the present invention, by “clone” or “clonal population” is meant a group of genetically identical cells derived from a single cell. The T cell receptor (or TcR) present at the surface of each of the T cells of the clonal population is identical and specifically recognizes a precise portion of a given antigen.

Helper T lymphocytes may be obtained from mononuclear cells of peripheral blood (PBMC) taken by means of a cytapheresis.

Activated helper T lymphocytes may be obtained by cultivating precursor cells in the presence of a source of antigen presenter cells, such as dendritic cells, and of suitable immunogenic peptides. Advantageously, antigen presenter cells may be made unable to multiply after irradiation (for example by exposure to about 5,000 rads). If necessary, nutritive cells (such as irradiated autologous B cells) may also be present. After a suitable incubation time (for example, about 7 to 28 days), during which the precursor cells are activated and proliferate as effector cells, the obtained cells, i.e. activated helper T lymphocytes, may be re-perfused into the individual from which they were taken, in order to induce an immune response against the human immunodeficiency virus.

According to another alternative, the thereby obtained cells may be re-perfused into an individual different from the one having provided the cells, but being compatible or made compatible at HLA level.

The antigen presenter cells may be dendritic cells, B lymphocytes or any other cells bearing HLA-DR1 molecules at the surface.

As an example of a method with which lymphocytes may be obtained and suitable for applying the invention, the method described in Patent Application WO 01/29190 may be mentioned.

Moreover, the antigen presenter cells may also be used as a means with which an immune response may be obtained. These cells may be loaded by means of peptides according to the invention and/or their functional analogs and/or multi-epitope constructs, and/or sequences of nucleic acids according to the invention.

Thus, the object of the present invention is further at least one isolated antigen presenter cell bearing at the surface, HLA-DR1 molecules, associated with at least one peptide and/or at least one nucleic acid according to the invention.

The antigen presenter cells suitable for applying the invention may for example be dendritic cells, B lymphocytes, macrophages, as well as the so-called artificial antigen presenter cells.

As an example of methods with which antigen presenter cells may be obtained, mention may be made of the techniques described in Patent Applications WO 97/44441, WO 03/089629, WO 93/02185 and WO 97/29182 or described by Brossart et al., (Blood, 1998, 92:4238-47).

Pharmaceutical Compositions

The peptides according to the present invention and the functional analogs thereof may also be used for preparing pharmaceutical compositions intended for preventing and/or treating an infection by a human immunodeficiency virus in HLA-DR1 patients. In particular, the human immunodeficiency virus may be the HIV-1.

According to a particular embodiment, the pharmaceutical composition advantageously is a vaccine.

The pharmaceutical compositions according to the present invention may comprise in combination with a pharmaceutically acceptable vehicle, excipient, diluent, and/or adjuvant, a peptide according to the invention, a functional analog thereof, a multi-epitope peptide as defined earlier, a sequence of nucleic acids as defined earlier, if necessary in an expression vector, an antibody as defined earlier, and mixtures thereof. These elements will be designated subsequently by the expression “active agent(s)”.

The excipients likely to be suitable for applying a pharmaceutical composition according to the invention may be e.g. buffering and pH adjustment agents, agents for adjusting the tonicity of the medium, wetting agents.

The pharmaceutical compositions according to the present invention may be administered via a local route, via an oral route, a topical route, or a parenteral route, such as the intravenous, sub-cutaneous, intradermal or intramuscular route for example.

Thus, the pharmaceutical compositions according to the invention may comprise an active agent as defined earlier, advantageously dissolved or suspended in a pharmaceutically acceptable sterile carrier, such as an aqueous medium for example.

The thereby obtained aqueous solution may then be conditioned for use as such or freeze-dried. The freeze-dried preparation is then combined with a sterile aqueous solution before its administration.

Within the scope of a therapeutic application, the compositions according to the invention may be administered to an individual already infected with HIV, in a sufficient amount in order to stop propagation of the virus in the organism or to at least partially stop the symptoms of the disease and its complications.

With regard to AIDS, the term “treatment” relates to a reduction in the severity of the disease, for example by reducing the disappearance of CD4+ T lymphocytes.

With regard to AIDS, the term “prevent” relates to the fact of preventing occurrence of AIDS, for example by administering a pharmaceutical composition according to the invention, before developing the disease. This indicates that a pharmaceutical composition according to the present invention may be used as a prophylactic agent in order to notably prevent reduction in the number of CD4+ T lymphocytes.

An adequate amount of one of the active agents with which this may be accomplished, is defined as an effective amount.

The effective amount of active agent should be adjusted according to the nature of said agent, the severity of the disease, the composition to be injected, the weight and the general condition of the patient.

As an example, for a patient of about 70 kg, the effective amount for example within the scope of a peptide according to the invention, may vary from about 100 μg/infection/day to about 3,000 μg/injection per day, and notably be of about 1,500 μg/injection per day in or several doses.

According to an embodiment, the administration method may comprise a primary injection, followed by one or more booster doses.

According to another alternative embodiment, the active agents as defined earlier may be associated with administration vectors (or carriers) such as liposomes, exosomes, or antigen presenter cells, as defined earlier (like dendritic cells or macrophages), or with a mixture thereof. The active agents associated with vectors may be positioned inside and/or at the surface of the latter.

Administration by means of liposomes may allow particular tissues, such as lymphoid tissues to be targeted by active agents. Advantageously, liposomes may also allow the half-life of the active agents to be increased.

In the pharmaceutical compositions comprising liposomes as an administration vector, the active agents may be incorporated into the liposomes, alone or in combination with a molecule promoting binding with e.g. lymphoid and/or myeloid cells, such as monoclonal antibodies which bind to surface markers, such as for example CD45, CD43 or CD86, or DC-SIGN or L-SIGN. Thus, the liposomes associated with active agents according to the invention, may be directed towards the tissues comprising lymphoid and/or myeloid cells and thereby release the active agent more effectively.

Many methods are known to one skilled in the art for preparing liposomes, such as for example the methods described in U.S. Pat. No. 4,235,971 or U.S. Pat. No. 4,837,028.

A liposome suspension may be administered via an intravenous route, locally, or topically according to an amount which notably depends on the active agent to be delivered and on the condition of the individual to be treated.

The active agent may also be administered as exosomes. Exosomes are vesicles of about 50 to 90 nm obtained from dendritic cells upon their maturation. These vesicles have a characteristic protein composition, notably comprising HLA molecules of type I and II, as well as other co-stimulation molecules, thereby providing activation of the innate and adaptive immune response.

The exosomes may be obtained from dendritic cells derived from mononuclear cells of peripheral blood, for example sampled by cytapheresis, and subsequently grown in a medium comprising IL4 and GM-CSF, or according to conditions defined in application WO 97/44441.

The exosomes suitable for applying the present invention may be prepared for example according to the method described in U.S. Pat. No. 6,812,023.

The object of the present invention is also the use of at least one active agent as defined earlier, such as a peptide and/or a nucleic acid according to the invention, for preparing a pharmaceutical composition intended for preventing and/or treating an infection by a human immunodeficiency virus, and in particular HIV-1, in patients with HLA-DR1 phenotype.

The present invention also relates to a method for treating and/or preventing an infection by a human immunodeficiency virus comprising at least one step consisting of administering to an individual an effective amount of at least one active agent as defined earlier such as a peptide and/or a nucleic acid according to the invention.

The peptide and/or the nucleic acid according to the invention applied in this method may be comprised in a pharmaceutical composition as defined earlier.

Diagnostic Method

According to another embodiment, the present invention also relates to a diagnostic method for determining the immune condition of an individual likely to have an infection by a human immunodeficiency virus, in particular HIV-1, comprising at least the steps of:
putting helper T lymphocytes isolated from said individual in contact in vitro with a peptide according to the present invention or a functional analog thereof, and
evaluating a biological response.

Putting isolated helper T lymphocytes in contact in vitro with a peptide according to the invention or functional analogs thereof, may be performed directly or by means of antigen presenter cells, as defined earlier.

According to an embodiment, the individual is of an HLA-DR1 phenotype.

The evaluated biological response may, for example, be a proliferation (or stimulation) index, secretion of one or more biological molecules in the extracellular medium or in the cytoplasm, the appearance and/or disappearance of one or more molecules associated with the cells, and a combination of these responses.

As an example, the biological responses obtained in helper T lymphocytes may be evaluated according to the methods described in the experimental part of the present text or in Patent Application WO 04/050909.

The biological molecules associated with the helper T lymphocytes able to be detected may for example be cytokines and/or chemokines and/or enzymes and/or surface determinants with which a phenotype of a population of particular helper T cells may be characterized. IFN-γγ, IL-2, IL-4, IL-5, IL-10 may be mentioned as an example of cytokines.

The biological molecules secreted by the helper T lymphocytes following their activation, may also be detected by means of the ELISPOT technique.

For example, IFN-γγ may be detected by means of an ELISPOT test, after incubation of T cells (either in the presence or not of other mononuclear cells of peripheral blood) with peptides according to the invention, as described in Forsthubert et al., (Sciences, 1996, 271:1728-30).

As an example of surface determinants, the appearance or disappearance (or the presence or absence) of which may be measured, mention may be made of CD4, CD28, CD69, CTLA-4, CD45-RA, CD45-RO, CD62-L.

Detection of surface determinants may be performed by any technique known to one skilled in the art, notably by flux cytometry, after marking the molecules to be detected by means of fluorescent probes, for example fluorescent antibodies.

As a biological response, it is also possible to measure the proliferation (or stimulation) index of the helper T lymphocytes after their being put in contact in vitro with one or more peptides according to the invention or functional analogs thereof.

Measurement of proliferation (or stimulation) may be performed for example by measuring the incorporation of [³H]-thymidine (as described in the experimental part of the present text) or by the method of dilution of a fluorescent probe, as described in application WO 04/050909, or by Lyons, (J. Immunol. Methods, 2000, 243:147-154) or Givan et al., (J. Immunol. Methods, 1999, 230:99-112).

The present invention also relates to a diagnostic method with which the immune condition of a subject likely to have an infection by a human immunodeficiency virus may be determined, comprising at least the steps consisting of quantitating in vitro, in a population of isolated lymphocytes from said subject, lymphocytes capable of being activated by putting them into contact with a complex obtaining an HLA-DRA molecule and a peptide according to the invention, or functional analogs of the latter.

This quantitation may be performed with or without a preliminary step for stimulating isolated lymphocyte cells from the subject.

Thus, after sampling T lymphocytes in a subject, detection may then be performed, for example by flux cytometry, by means of antibodies marked with a fluorescent probe and specific to this receptor.

According to another alternative, it is possible to detect these helper T lymphocytes by means of a tetramer, for example as described earlier, marked with a fluorescent probe, for example according to the procedure described by Novak et al., J. Clin. Invest., 1999, 104:R63-R67, or by Cochran et al., Immunity, 2000, 12:241-250.

The present invention also relates to a diagnostic kit with which the methods described earlier may notably be applied and comprising at least one peptide according to the present invention or functional analogs thereof.

The present invention also relates to a diagnostic kit with which the methods described earlier may notably be applied and comprising at least one tetramer as defined earlier.

The invention described herein is liable to variations and modifications. The invention includes these variations and modifications.

The invention may be better understood upon reading the following examples. However the latter should not be interpreted as limiting the scope of the invention.

### CAPTIONS OF THE FIGURES

FIG. 1: The stimulation index of CD4+ T lymphocytes stemming from Class II H-2, invalidating HLA-DR1 transgenic mice (IAβ^{bo}) and immunized by means of the p24 protein of the HIV, is illustrated in this figure. The CD4+ T cells were re-stimulated in vitro by blasts activated by LPS and loaded with peptide Gag₂₉₁₋₃₁₀, Gag₃₀₁₋₃₂₀, Gag₃₂₁₋₃₄₀, Gag₃₃₁₋₃₅₀, and RT₁₇₁₋₁₉₀ as control peptide. The stimulation index represents the ratio of the radioactivity measured after incorporation of [³H]-thymidine by the T lymphocytes in the presence of specific peptides over the radioactivity measured by incorporation of [³H]-thymidine by the T lymphocytes in the presence of the control peptide.

FIG. 2: The stimulation indexes for CD4+ lymphocytes isolated from seronegative HLA-DR1 patients and initiated by dendritic cells loaded with the p24 antigen of HIV-1 (FIG. 2A) or MN HIV-1 inactivated by aldrithiol-2 (MNAT2) (FIG. 2B). The cells were then stimulated by the Gag₃₃₁₋₃₅₀ peptide or the SM 28GST₁₉₀₋₂₁₁ control peptide.

FIG. 3: Table 1: The sequences of the Gag₃₀₁₋₃₂₀, Gag₃₂₁-340, Gag₃₃₁₋₃₅₀, and Gag₂₉₁₋₃₁₀ epitopes as well as their frequency in HIV-1 viruses of the B group are illustrated in this Table.

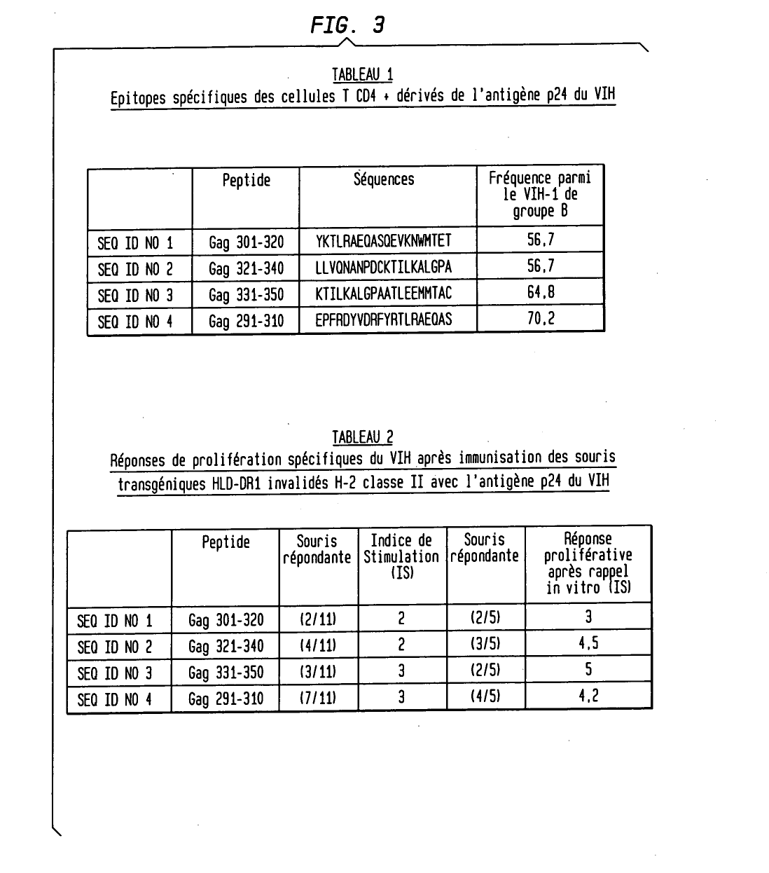

Table 2: The number of mice responding to the Gag₃₀₁₋₃₂₀, Gag₃₂₁₋₃₉₀, Gag₃₃₁₋₃₅₀, and Gag₂₉₁₋₃₁₀ epitopes as well as the intensity of the observed proliferation responses is illustrated in this Table.

FIG. 4: Table 3: Responses with secretion of IFN-γγ by CD4+ T cells isolated from HLA-DR1 patients infected by HIV-1 are illustrated in this Table. The responses of specific human CD4+ T cells of HIV-1 were evaluated by an ELISPOT test as described in the section “Materials and Methods”. The responses were evaluated with or without eliminating CD8+ T cells. The peptides used for stimulating PBMCs were either a set of peptides overlapping in length by 15 amino acids (set of 11 consecutive peptides), or a peptide of 20 amino acids in length.

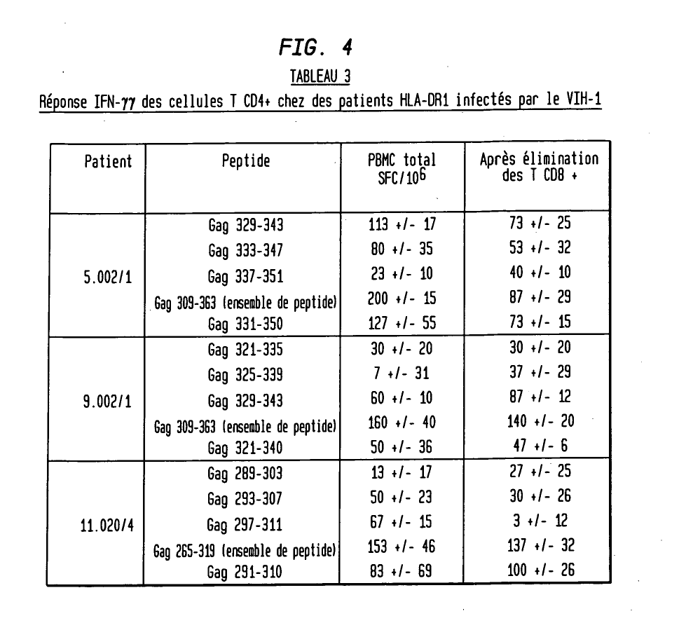

### MATERIALS AND METHODS

### Sequence Analysis

The Gag protein of the HIV-1 was analyzed by means of the TEPITOPE program (Hammer et al., Adv. Immunol., 1997, 66:67-100) in order to search for the presence of peptide sequences with a length of 20 amino acids containing a pattern for binding to the HLA-DR1 molecule. The prediction threshold was set to 4%.

Peptide Synthesis

Peptides overlapping by 15 and 20 mers covering the whole of the Gag molecule were synthesized and provided by the National Institute of Heath—NIH USA. The peptides were placed in solution in an amount of 1 mg/ml in a PBS (Phosphate Buffered Saline) buffer containing 10% DMSO.

Transgenic Mouse

HLA-DR1 transgenic and Class II (IAβ^{bo}) H-2 invalidated mice, the line of which was established beforehand by Pajot et al., (Int. Immunol., 2004, 16:1275-1282) were used during the various experiments.

Immunization and Proliferation Tests

The p24 recombinant protein of HIV-1 used during the various experiments was obtained from ABCYSS (Paris, France).

The mice, anesthetized with 75 mg/kg of pentobarbital (Ceva, Santé Animale, Libourne, France) received 4 μg of recombinant proteins associated with an alum adjuvant.

Twelve days after the last immunization, splenocytes separated from red corpuscles by purification on Ficoll (5·10⁶ cells/25 cm² of culture flask (TECHNO PLASTIC PRODUCT, TPP, Trasadingen, Switzerland)) were co-cultivated in the presence of blast cells activated with LPS (5·10⁶ cells/culture flask), loaded with peptides (20 μg/ml) and irradiated by γ rays (180 Gy), in an RPMI medium complemented with 10% FCS (Fetal Calf Serum), 10 mM HEPES, 1 mM sodium pyruvate, 5·10⁵ M mercapto-2-ethanol, 100 IU/ml of penicillin and 100 μg of streptomycin, as described by Loirat at al., (Journal of Immunology, 2000, 165:4748-4755).

The blast cells were loaded with one of the test peptides Gag₃₀₁₋₃₂₀, Gag₃₂₁₋₃₄₀, or Gag₃₃₁₋₃₅₀, or with a negative control peptide derived from an antigen of the HIV, the RT₁₇₁₋₁₉₀ peptide, or with a positive control peptide derived from the p24 antigen of the HIV, the peptide Gag₂₉₁₋₃₁₀, respectively.

After seven days of culture, proliferation tests were carried out after distributing 5·10⁵ cells/wells, in 96-well plates with flat bottoms, from cells (obtained from TTP), in the presence of blast cells stimulated by LPS, irradiated and loaded with peptide (in an amount of 2·10⁵ cells/well). The proliferation tests were conducted over a period of 72 hours, in a full RPMI medium complemented with 3% SVF.

The cells were incubated for the last 16 hours, with 1 μCi of [³H]-thymidine per well before being recovered on filters with a TOMTEC collector (Perkin Elmer Applied Biosystem). The incorporated radioactivity was measured with a micro-β counter (Perkin Elmer Applied Biosystem)

The results are expressed as a stimulation index (IS) equal to the ratio of the cpm measured after incubation with the tested peptides over the cpm measured after incubation with the control peptide.

Population Study

HIV-1 seronegative individuals were selected with regard to their HLA-DR1 profile.

The individuals infected by HIV-1 were selected from the French cohort ALT and selected with regard to their HLA-DR1 profile. The long term non-progressor condition of the patients was defined as being an asymptomatic HIV infection for at least 8 years, with a stable CD4+ T cell count at a level larger than or equal to 600 cells/mm³, and without any anti-retrovirus therapy (Candotti at al., J. Med. Virol. 1999, 58:256-63).

Elispot Test

HIV-1-specific human CD4+ T cell response towards various peptides was quantitated by the ELISPOT test on frozen mononuclear cells of peripheral blood (PBMC: peripheral blood mononuclear cells), as described earlier (Forsthubert et al., Science, 1996, 271:1728-30).

ELISPOT 96-well plates (Millipore, Molsheim, France) were covered with an antibody directed against human IFN-γγ (IgG1/B-B1, Diaclone, Strasburg, France).

After a blocking step in the presence of a medium containing 10% SVF, 1·10⁵ PBMCs were distributed into the wells. The experiment was conducted in triplicate. The cells were grown during the night following their thawing. Next, the plates were incubated at 35° C. for 18 hours in the presence of 2 μg/ml of peptides derived from the Gag protein of HIV-1 (a 15-mer or 20-mer overlapping peptide; cf. Table 3).

Phytohemagglutinin (Murex, Paris, France, 1 μg/nl) and the medium alone were used as a positive and a negative control, respectively.

The wells were then subsequently washed and spots were detected by adding the detection antibody directly against human IFN-γγ and marked with biotin (B-G1-Diaclone) (4 hours, 37° C.), followed by adding the alkaline phosphatase on the streptavidin (1 hour, 37° C.) and the substrate (5-bromo-4-3-indolyl-phosphate/4-nitro-blue-tetrazolium; Sigma, Saint-Quentin Falavier, France). Incubation was maintained at room temperature, until blue dots appeared. The frequency of spot forming cells (SFCs) specific to the antigens was measured by means of an automated microscopy system (ZEISS, Munich, Germany) and counted as positive if a minimum of 50 SFCs/1·10⁶ of PBMCs were detected above the background noise. The ELISPOT tests were repeated after removing the CD8+ T cells by means of anti-CD8 magnetic beads (Dynabeads CD8, Dynal). The fact that more than 99.8% of the CD8+ cells were removed from the PBMCs, was able to checked by flux cytometry (EPICS, XL COULTER).

Preparation of Dendritic Cells (DC) and Loading with Antigens

The dendritic cells were prepared from PBMCs such as described earlier by Brossart et al., (Blood, 1998, 92:4238-47). The PBMCs were isolated from the blood of HLA-DRB1*01⁺ healthy donors by centrifugation on a Ficoll-Hypaque gradient.

The PBMCs were cultivated in 6-well plates (10-15·10⁶ cells/wells and left to adhere for 1 hour at 37° C. The non-adherent cells were removed and the adherent monocytes were cultivated in RPMI containing 10% FCS, penicillin/streptomycin (50 units/ml-50 μg/ml), 10 mM Hepes, 10 mM glutamine, 1,000 units/ml of IL-4 (R & D, France) and 500 units/ml of GM-CSF (Leucomax, Aventis, France). The DCs of the preparation have a phenotype of immature cells: CD1a⁺, CD4a⁺, CD14⁻, low CD83⁺, low CD86⁺, HLA-DR⁺, DC-SIGN⁺. Subsequently, the DCs were frozen (in PBS containing 4% of human albumin, LFB, France) or loaded with antigens.

The DCs were loaded with the recombinant p24 protein of HIV (10 μg/ml, Sigma, France) or of MN HIV-1 inactivated by aldrithiol-2 (AT-2) (1,000 ng of p24/ml) with maturation agents Ribomunyl® (1 μg/ml) and IFN-γγ (500 units/ml) for two hours at 37° C.

T Cell Initiation

The CD4+ T cells were isolated from the non-adherent fraction of the PBMCs by negative removal and using magnetic beads (Miltenyl Biotec, France). They were kept frozen, and then thawed before stimulating the T cells.

The purified CD4+ T cells were incubated in the presence of irradiated (5,000 rads) mature autologous DCs, loaded with antigens (in a ratio of 5/1), as well as in the presence of irradiated (5,000 rads) autologous B cells (in a ratio of 1/2) in RPMI medium containing 10% human serum (Institut Jacques BOY, France) containing non-essential amino acids (GIBCO, France), 1 mM sodium pyruvate (GIBCO, France), 10 mM Hepes, 10 mM glutamine.

Some rhIL-2 in an amount of 100 units/ml (Proleukin, Chiron, France) was added 5 days after the stimulation.

Ten days after stimulation, the T cell cultures were re-stimulated with irradiated DCs in a ratio of 1/20, loaded with the Gag₃₃₁₋₃₅₀ peptide of the SM 28GST₁₉₀₋₂₁₁ control peptide (190-211 epitope of the antigen of Shistosoma mansoni 28 kDa glutathione S-transferase) as well as in the presence of autologous nutritive cells according to a ratio of 1/5.

The CD4+ T cell cultures were subsequently re-stimulated every 10-15 days with autologous B cells transformed with EBV (Epstein-Bar Virus, EBV B cells), irradiated and loaded with peptides, according to a ratio of 1/2, and of irradiated allogenic PBMCs according to a ratio of 1/4.

Human T Cell Proliferation Test

The T cells in cultures were put into contact with the Gag₃₃₁₋₃₅₀ peptide or the SM 28GST₁₉₀₋₂₁₁ peptide in an amount of 50 μg/ml for 3 hours at 37° C., and then distributed into 96-well flat-bottom plates (5·10⁵ cells/well). The cells were cultivated in medium for T cells in the absence of rhIL-2 for 3 days before being pulsed with 1 μCi[³H]-thymidine for 20 hours.

The cells were recovered on filters by using a TOMTEC collector (Perkin Elmer Applied Biosystems) and the incorporated radioactivity was measured with a micro-β counter (Perkin Elmer Applied Biosystems).

The results are expressed as a stimulation index (SI) representing the ratio of the counts measured after incubation with the tested peptide over the counts measured after incubation with the control peptide.

### Example I

### Identification and Antigenicity of New HLA-DR1 Epitopes in HLA-DR1 Transgenic Mice

In order to identify novel peptides with HLA-DR1 specificity, the sequence of amino acids of the Gag protein was scanned in order to search for HLA-DR1 specific patterns by means of the TEPITOPE algorithm.

Peptides with 20 amino acids containing a core region of 9 amino acids comprising an HLA-DR1 pattern and additional N- and C-terminal amino acids were selected, among which are found three novel peptides Gag₃₀₁₋₃₂₀, Gag₃₂₁₋₃₄₀, Gag₃₃₁₋₃₅₀, and a known peptide Gag₂₉₁₋₃₁₀ (Iyasere et al., J. Virol., 2003, 77:10900-9) (Table 1). Table 1 also indicates the frequency with which the identified sequences are present in the HIV-1 strain of the B group. This frequency, expressing the preservation percentage is determined on the number of HIV strains which have the same sequence of amino acids belonging to the B group (HIV databases, Los Alamos National Library and National Institutes of Health, hhtp://hiv-web.lan1.gov/cgi-bin/EPILIGN/epilign.cgi).

In order to evaluate the immunogenicity of HLA-DR1 restrictive epitopes, Gag₃₀₁₋₃₂₀, Gag₃₂₁₋₃₄₀, and Gag₃₃₁₋₃₅₀, the Class II H-2 invalidated and HLA-DR1 transgenic mice were immunized with the p24 protein of the HIV. Subsequently, the T cells from the spleen were re-stimulated in vitro with the peptides to be tested and a positive control peptide Gag₂₉₁₋₃₁₀, or a negative control peptide RT₁₇₁₋₁₉₀.

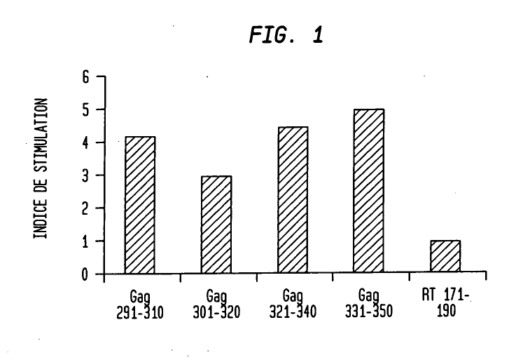

FIG. 1 shows that the Class II H-2 invalidated and HLA-DR1 transgenic mice are capable of developing a proliferative cell response against restricted HLA-DR1 Gag₂₉₁₋₃₁₀ peptides, identified previously by Iyasere et al., (J. Virol. 2003, 77:10900-9) as being an HLA-DR1 restricted immuno-dominant peptide capable of inducing a response in CD4+ T cells isolated from HLA-DR1 individuals infected with HIV, as well as against the Gag₃₀₁₋₃₂₀, Gag₃₂₁₋₃₄₀, and Gag₃₃₁₋₃₅₀ peptides.

No response was induced in CD4+ T cells stimulated by means of the control peptide derived from the RT antigen of HIV-1.

Table 2 shows that two Class II H-2 invalidated and HLA-DR1 transgenic mice out of 11 respond to Gag₃₀₁₋₃₂₀ peptides, 4 mice out of 11 respond to the Gag₃₂₁₋₃₄₀ peptides and 3 mice out of 11 respond to the Gag₃₃₁₋₃₅₀ peptides with a stimulation index from 2 to 3.

Further, by an additional booster dose in vitro with these peptides, the proliferation index may be increased from 3 to 5, thereby confirming the specificity of the response.

### Example II

### Recognition of the Gag₃₂₁₋₃₄₀ and Gag₃₃₁₋₃₅₀ Epitopes of HIV-1 p24 by HLA-DR1+ Patients Infected by HIV

Recognition of the epitopes identified by CD4+ cells isolated from HLA-DR1+ patients infected by HIV-1 was evaluated by using the ELISPOT test. PBMCs were isolated in a group of patients all responding to the whole p24 antigen and/or to a set of 15-mer overlapping peptides covering the p24 protein (Table 3).

Thus, as shown by Table 3, the epitope Gag₂₉₁₋₃₁₀, described earlier as an HLA-DR1 epitope is capable of stimulating the CD4+ T cells in the 11.020/4 patient and of inducing a positive and unaltered response after removal of the CD8+ cells.

The Gag₃₃₁₋₃₅₀ peptide is recognized by the CD4+ T cells of the 5.002/1 patient as well as the set of 15-mer peptides comprising the Gag₃₃₁₋₃₉₀ epitope.

The Gag₁₂₁₋₃₄₀ peptide induces a response at the detection threshold limit by the CD4+ T cells in patient 9.002/1, which also recognize the set of the peptides with the same order of magnitude.

Thus, both novel HLA-DR1 restrictive epitopes, Gag₃₂₁₋₃₄₀ and Gag₃₃₁₋₃₅₀, which are immunogenic in the Class II H-2 invalidated HLA-DR1 transgenic mouse may also induce a response in HIV-infected HLA-DR1 patients.

### Example III

### Stimulation of CD4+ Cells Isolated from a Seronegative HLA-DR1 Individual by the Epitope Gag₃₃₁₋₃₅₀

The PBMCs of an HIV-1 seronegative HLA-DR1 individual (BRE individual) were isolated and initiated in vitro with autologous DCs (loaded with the p24 protein of the HIV or the entire MN virus inactivated by aldrithiol-2 (MNAT2)) and the CD4+ T cells were separated from the other cells and cultivated.

The CD4+ T cells were then cultivated in vitro for 2 weeks in the presence of antigen presenter cells, irradiated autologous EBV B cells and loaded by means of the p24 peptide of the HIV or MNAT2 virus.

Next, the CD4+ T cells were tested with regard to a specific proliferative response induced by the peptide Gag₃₃₁₋₃₅₀ or the control peptide SM 28GST₁₉₀₋₂₁₁.

After three days of incubation with the antigen, incorporation of [³H]-thymidine was measured. A stimulation index larger than 2 was considered as being significant.

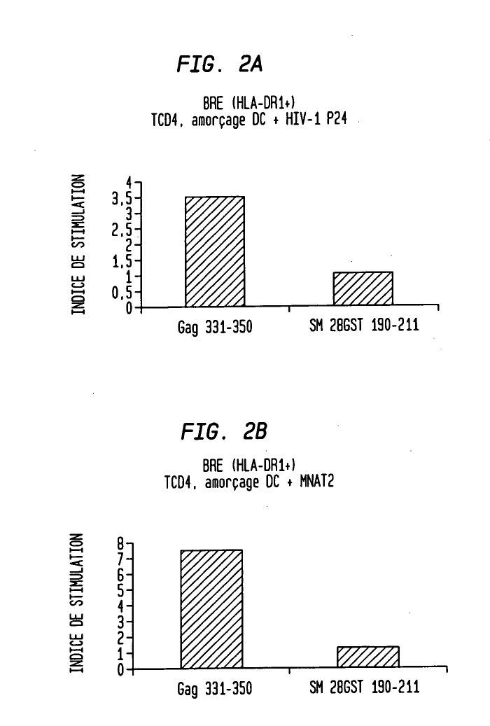

As shown in FIG. 2, the CD4+ T cells of the BRE individual have proliferated in response to the Gag₃₃₁₋₃₅₀ peptide, but no response was observed in the presence of the control peptide SM 28GST₁₈₈₋₂₁₁ or of the medium alone. The stimulation index is equal to 10.

Thus, the Gag₃₃₁₋₃₅₀ epitope, also immunogenic in Class II H-2 invalidated and HLA-DR1 transgenic mice is capable of initiating a response of CD4+ T cells in an HIV-1 seronegative HLA-DR1 donor.

1. A peptide comprising an epitope consisting in a sequence of amino acids selected from SEQ ID NO 1, SEQ ID NO 2, SEQ ID NO 3, said peptide being associated with a biotinylated complex consisting of an α chain (DRα) and of β chain (DRβ) of an HLA of type II, of a spacer arm, and of streptavidin in order to form a Class II tetramer.

2. An antibody capable of specifically binding to an epitope consisting in a sequence of amino acids selected from SEQ ID NO 1, SEQ ID NO 2, SEQ ID NO 3 or to a complex comprising said epitope and an HLA-DR1 molecule.

3. The use of at least one peptide comprising at least one epitope consisting in a sequence of amino acids selected from SEQ ID NO 1, SEQ ID NO 2, SEQ ID NO 3, and/or a nucleic acid coding for said sequence in order to prepare a pharmaceutical composition intended for preventing and/or treating an infection by a human immunodeficiency virus in patients of HLA-DR1 phenotype.

4. The use according to the preceding claim, wherein the peptide and/or the nucleic acid is associated with a carrier selected from a liposome, an exosome, and an antigen presenter cell bearing HLA-DR1 molecules at the surface.

5. The use according to the preceding claim, wherein the antigen presenter cell is a dendritic cell or a macrophage.

6. An isolated antigen presenter cell bearing at the surface, HLA-DR1 molecules, associated with at least one peptide comprising at least one epitope consisting in a sequence of amino acids selected from SEQ ID NO 1, SEQ ID NO 2, SEQ ID NO 3 and/or a nucleic acid coding for said sequence.

7. An isolated helper T lymphocyte clone, comprising a receptor which specifically recognizes a complex containing an HLA-DR1 molecule and a peptide comprising an epitope consisting in a sequence of amino acids selected from SEQ ID NO 1, SEQ ID NO 2, SEQ ID NO 3.

8. A diagnostic method for determining the immune condition of an individual likely to have an infection by a human immunodeficiency virus comprising at least steps of:
putting helper T lymphocytes isolated from said individual in contact in vitro with at least one peptide comprising at least one epitope consisting in a sequence of amino acids selected from SEQ ID NO 1, SEQ ID NO 2, SEQ ID NO 3 and
evaluating a biological response.

9. The method according to the preceding claim, wherein the biological response is selected from proliferation, secretion of biological molecules, appearance and/or disappearance of molecules associated with said cells, and a combination of these responses.

10. A diagnostic method in order to determine the immune condition of an individual likely to have an infection by a human immunodeficiency virus comprising at least the step consisting of quantitating in vitro, lymphocytes isolated from said individual, capable of being activated by putting them into contact with a complex containing an HLA-DR1 molecule and a peptide comprising an epitope consisting in a sequence of amino acids selected from SEQ ID NO 1, SEQ ID NO 2, SEQ ID NO 3.

11. The method to the preceding claim, wherein the quantitation step comprises the use of tetramers as defined according to claim 1.

12. A diagnostic kit comprising at lest one peptide comprising at least one epitope consisting in a sequence of amino acids selected from SEQ ID NO 1, SEQ ID NO 2, SEQ ID NO 3.

13. A diagnostic kit comprising at least one tetramer as defined according to claim 1.


SEQUENCE LISTING (4 sequences)

SEQ ID NO: 1 - protein, 20 aa, Human immunodeficiency virus
  CHAIN (1)..(20)
YKTLRAEQASQEVKNWMTET

SEQ ID NO: 2 - protein, 20 aa, Human immunodeficiency virus
  CHAIN (1)..(20)
LLVQNANPDCKTILKALGPA

SEQ ID NO: 3 - protein, 20 aa, Human immunodeficiency virus
  CHAIN (1)..(20)
KTILKALGPAATLEEMMTAC

SEQ ID NO: 4 - protein, 20 aa, Human immunodeficiency virus
  CHAIN (1)..(20)
EPFRDYVDRFYRTLRAEQAS